(19) United States
(12) Patent Application Publication
(10) Pub. No.: US 2011/0027171 A1
(43) Pub. Date: Feb. 3, 2011

(54) PH SENSITIVE LIPOSOME COMPOSITION

(76) Inventor: Sofou Stavroula, Brooklyn, NY (US)

Correspondence Address: SMITH, GAMBRELL & RUSSELL, SUITE 3100, PROMENADE II, 1230 PEACHTREE STREET, N.E., ATLANTA, GA, 30309-3592, US

(21) Appl. No.: 12/443,496

(22) PCT Filed: Oct. 5, 2007
(86) PCT No.: PCT/US07/80614

§ 371 Date: Oct. 18, 2010

Related U.S. Application Data
(60) Provisional application No. 60/828,523, filed on Oct. 6, 2006.

(51) Int. Cl. A61K 9/127; A61K 51/12; A61K 31/7088; A61K 38/00; A61K 39/395; A61P 35/00

(52) U.S. Cl. 424/1.21; 424/450; 514/44 R; 514/1.1; 424/178.1

(57) ABSTRACT

Using liposomes to deliver bioactive agents to cancer or tumor cells and compositions of specific lipids that form liposomes to deliver a biologically active agent.

### STATEMENT OF RELATED APPLICATIONS

This application is based on and claims priority on U.S. Provisional Patent Application No. 60/828,523 having a filing date of 6 Oct. 2006.

### BACKGROUND

1. Technical Field

This invention generally relates to the field of therapeutic delivery systems and liposome compositions. Further, this invention is directed to a composition of specific lipids that form liposomes, which can deliver a biologically active agent.

2. Related Art

Lipidic particles can be complexed with virtually any biological material. This capability allows these lipidic particles to be used as delivery systems for bioactive agents. Lipidic complexes have been used for a myriad of drug therapies, and one area in which these delivery systems have shown promising results is in cancer therapies. For a cancer therapy to be successful and efficient, the bioactive agent should be targeted to the tumor or cancer cell.

In some cases, only after the drug carriers are localized within the tumor interstitium, cancer-targeting ligands are necessary to enhance binding of the carriers to cancer cells, and to mediate their cellular internalization that increases drug bioavailability. Kirpotin, D. B., et al., “Antibody Targeting of Long-Circulatin Lipidic Nanoparticles does not increase Tumor Localization but does increase Internalization in Animal Models”, Cancer Res., 66(13): 6732-6740 (2006). At other times, during circulation in the bloodstream, ‘decoration’ of the carrier surface with tumor-binding ligands can activate non-desirable interactions with the host's immune- and reticuloendothelial-(RES) systems resulting in fast removal of the carriers from the blood stream, in low tumor absorbed doses, and in accumulation of drug carriers in healthy organs where release of therapeutic contents will kill healthy cells and increase toxicity. Allen, T. M., “Ligand-Targeted Therapeutics in Anticancer Therapy”, Nature Cancer Rev., 2: 750-763 (2002).

Accordingly, there is always a need for an improved liposome for delivering bioactive agents. Additionally, there is always a need for an improved liposome that can be targeted to tumors and cancer cells. Further there is a need for an improved liposome that can be minimally recognized by the reticuloendothelial and immune systems. It is to these needs, among others, that this invention is directed.

### BRIEF SUMMARY

Briefly, this invention relates to liposomes that are able to be tuned to ‘hide’ (or ‘mask’) the targeting ligands during circulation and to ‘expose’ the targeting ligands after the liposomes extravasate into the (acidic) tumor interstitium where the liposomes are in the close vicinity of cancer cells. These liposomes can effectively address the issue of toxicity of immunoreactivity. This invention includes types of liposomes that can circulate for longer periods of time in the blood stream and can be absorbed by tumors after accumulation within the tumor interstitium, to result in internalization by solid-tumor cancer cells with less identification by the immune- and RES-systems. These liposomes can exhibit high tumor accumulation and high drug bioavailabilty in vivo within tumor cells. In one embodiment, the liposomes can comprise ionizable ‘domain-forming’ (‘raft’-forming) rigid lipids that are triggered to form lipid-phase separated domains in response to the tumor interstitial acidic pH (e.g. 6.7) environment. The liposomal membrane can be composed of rigid lipids and PEGylated lipids so as to increase the blood circulation times. Further, PEGylation may not interfere with the pH-sensitive properties of the developed liposomes, as the domain-forming property of rigid-lipids (each being lamellar-forming) can be utilized.

Tumor-targeting ligands can be conjugated on the headgroup of ‘raft’-forming lipids that preferentially partition into one type of domain after lipid-phase separation occurs at pH=6.7. Liposomes also contain PEGylated lipids that do not preferentially partition into the above mentioned domains after their formation. For example, at physiological pH of about 7.4 (e.g. in blood circulation) the lipids can be charged, the lipids composing the liposome membrane are ‘mixed’ on the plane of the membrane and are largely homogeneous, and the PEGylated lipids are uniformly distributed throughout the liposome membrane, thus adequately ‘masking’ (e.g. sterically hindering) the surface conjugated tumor-targeting ligands. As the pH is lowered, separated lipid domains are formed, in some of which tumor-targeting ligands are clustered and from which PEGylated lipids are excluded. As a result, the surface-conjugated ligands can be exposed with selectivity.

Using liposomes with targeting ligands that become ‘hidden’ or ‘exposed’ depending on the pH of their immediate environment, the fraction of liposomes that is internalized by cancer cells in vivo, after liposome extravasation into the tumor interstitium, can be dramatically increased within the cancer cells that constitute the metastatic vascularized tumors. This can allow for lower administered doses, and higher tumor adsorbed doses, which can result in lower toxicities.

These features, and other features and advantages of the present invention, will become more apparent to those of ordinary skill in the relevant art when the following detailed description of the preferred embodiments is read in conjunction with the appended drawings.

### BRIEF DESCRIPTION OF THE DRAWINGS

FIG. 1 is a diagrammatical illustration of the pH-tunable domain forming lipids that aggregate as the pH of an environment becomes more acidic.

FIG. 2 is a diagrammatical illustration of the pH-tunable domain forming lipids that ‘hide’ or ‘expose’ the surface conjugated targeting ligands depending on the pH of the environment.

FIG. 3 is a graph illustrating the extent of the binding of biotinylated liposomes with 0.01% mole of PEGylated lipids to streptavidin-covered microbeads at various pH levels.

FIG. 4 is a graph illustrating the extent of the binding of biotinylated liposomes with 0.25% mole of PEGylated lipids to streptavidin-covered microbeads at various pH levels.

FIG. 5 is a graph illustrating the extent of the binding of biotinylated liposomes with 0.5% mole of PEGylated lipids to streptavidin-covered microbeads at various pH levels.

FIG. 6 is a graph illustrating the extent of the binding of biotinylated liposomes with 0.75% mole of PEGylated lipids to streptavidin-covered microbeads at various pH levels.

FIG. 7 is a graph illustrating the extent of the binding of biotinylated liposomes with 1.0% mole of PEGylated lipids to streptavidin-covered microbeads at various pH levels.

FIG. 8 is a graph illustrating the extent of the binding of biotinylated liposomes with 1.5% mole of PEGylated lipids to streptavidin-covered microbeads at various pH levels.

FIG. 9 is a diagrammatical illustration showing that the pH-tunable domain forming lipids that ‘hide’ or ‘expose’ the surface conjugated targeting ligands depending on the pH of the environment cannot specifically bind to targets when the ‘effective lengths’ of the PEG-linked lipids are longer than the size of the targeting ligands.

FIG. 10 is table showing the calculated fractional increase in biotinylated-liposome binding between pH 7.4 and pH 6.5 for the lipid compositions ranging from 0.1 to 1.5% mole of total lipid.

FIG. 11 is a thermograph prepared from differential scanning calorimetry data showing the effect of pH on domain formation due to protonation of DSPS lipids.

FIG. 12 is a graph showing the percentage of calcein retention as a function of pH by liposomes composed of equimolar DPPC and DSPS over 5 days.

### DEFINITIONS

Unless specifically indicated otherwise, all technical and scientific terms used herein have the same meaning as commonly understood by those of ordinary skill in the art to which this invention belongs. For purposes of the present invention, the following terms are defined.

The term “cancer” as used herein refers to a disease of inappropriate cell proliferation and is more evident when tumor tissue bulk compromises the function of vital organs. Concepts describing normal tissue growth are applicable to malignant tissue because normal and malignant tissues can share similar growth characteristics, both at the level of the single cell and at the level of the tissue.

The term “anionic liposome” as used herein is intended to encompass any liposome as defined below that is anionic. The liposome is determined as being anionic when present in physiological pH. It should be noted that the liposome itself is the entity that is being determined as anionic. The charge and/or structure of a liposome of the invention present within an in vivo environment has not been precisely determined. However, in accordance with the invention an anionic liposome of the invention will be produced using at least some lipids that are themselves anionic. The liposome need not be comprised completely of anionic lipids but must be comprised of a sufficient amount of anionic lipid such that when the liposome is formed and placed within an in vivo environment at physiological pH the liposome initially has a negative charge.

A “pH-sensitive” lipid as used herein refers to a lipid whose ability to change the net charge on its head group depends at least in part on the pH of the surrounding environment.

“Biologically active agents” as used herein refers to molecules which affect a biological system. These include molecules such as proteins, nucleic acids, therapeutic agents, vitamins and their derivatives, viral fractions, lipopolysaccharides, bacterial fractions, and hormones. Other agents of particular interest are chemotherapeutic agents, which are used in the treatment and management of cancer patients. Such molecules are generally characterized as antiproliferative agents, cytotoxic agents, and immunosuppressive agents and include molecules such as taxol, doxorubicin, daunorubicin, vinca-alkaloids, actinomycin, and etoposide.

“Effective amount” as used herein refers to an amount necessary or sufficient to inhibit undesirable cell growth, e.g., prevent undesirable cell growth or reduce existing cell growth, such as tumor cell growth. An effective amount can vary depending on factors known to those of ordinary skill in the art, which include the type of cell growth, the mode and regimen of administration, the size of the subject, the severity of the cell growth. One of ordinary skill in the art would be able to consider such factors and make the determination regarding the effective amount.

“Liposome” as used herein refers to a closed structure comprising an outer lipid bi- or multi-layer membrane surrounding an internal aqueous space. In particular, the liposomes of the present invention form vase-like structures which invaginate their contents between lipid bilayers. Liposomes can be used to package any biologically active agent for delivery to cells.

The following abbreviations are used herein: PEG: polyethylene glycol; mPEG: methoxy-terminated polyethylene glycol; Chol: cholesterol; DTPA: diethylenetetramine pentaacetic acid; DPPC: dipalmitoylphosphatidylcholine; DSPA: distearoylphosphatidic acid; and DSPS: distearoylphosphatidylserine.

### DETAILED DESCRIPTION

Embodiments of this invention include pH-sensitive liposomes of a specific composition forming a stable structure that can efficiently carry biologically active agents. More particularly, the liposome can contain one or more biologically active agents, which can be administered into a mammalian host to effectively deliver its contents to and target a target cell or tumor cell. The liposomes can be capable of carrying biologically active agents such that the agents are sequestered in one environment and can be selectively exposed in another. Specifically, the use of a pH-tuned domain-forming membrane allows for tunable rigid-liposomes that can efficiently ‘expose’ the otherwise ‘hidden’ tumor-targeting ligands after liposome extravasation into tumors.

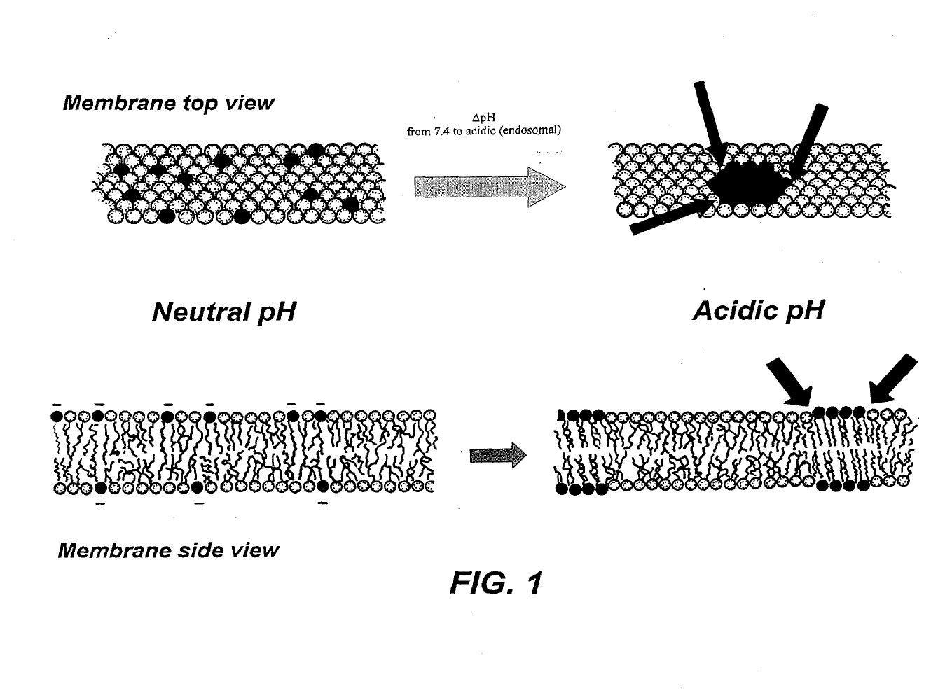

One aspect of this embodiment is to use pH-tuned liposomes as a mechanism to more efficiently and selectively expose the targeting ligand to the cancer cells composing a tumor or solid tumor. As shown in FIG. 1, which is a top and side view of pH-tunable liposomal membranes containing domain-forming lipids, the lipid-membrane surface appears homogeneous (mixed) at physiological pH (left) when electrostatic repulsion among the titratable anionic headgroups of domain-forming lipids is dominating (negative charges). At acidic pH (e.g. tumor interstitium 6.7) (right) protonation of the negatively charged headgroups allows the attractive Van der Waals forces among the hydrocarbon tails to dominate and lipid-separation and domain formation to occur. In general, under ‘raft’ or ‘domain’ hypothesis and as shown in FIG. 1, the long-saturated hydrocarbon-chains of phospholipids in membranes phase separate (aggregate in an ordered phase domain) in the plane of the membrane that should also contain lipids with unsaturated hydrocarbon-chains.

### Liposome Composition

One embodiment of this invention is a pH-sensitive liposome composition for targeting a biologically active agent to tumor cells, comprising:

a) at least two lipid phase separated domains formed by
i) a first lipid having a head group and a hydrophobic tail that, when protonated, is substantially miscible, wherein the first lipid is a zwitterionic lipid;
ii) a second lipid having a titratable charged head group, and a hydrophobic tail that, when protonated, is substantially immiscible with the first lipid;

b) a targeting ligand capable of binding an antigen or a marker and linked to the head group of a third lipid having a tail matching at least a portion of the first lipid or the second lipid,

wherein the liposome composition is adapted to laterally separate, via lipid phase separation, when the liposome composition is exposed to a specific environment, whereby the phase separation of the lipids exposes the targeting ligand to the more acidic environment.

Another embodiment of this invention is a liposome composition containing a biologically active agent, comprising:

a) at least two lipid phase separated domains formed by
i) a first lipid having a head group and a hydrophobic tail that, when protonated, is substantially miscible, wherein the first lipid is a zwitterionic lipid;
ii) a second lipid having a titratable charged head group, and a hydrophobic tail that, when protonated, is substantially immiscible with the first lipid;

b) a third lipid having a tail matching at least a portion of the first lipid or the second lipid, wherein the third lipid is PEG-linked; and

c) a targeting ligand capable of binding an antigen or a marker and linked to the headgroup of a fourth lipid having a tail matching at least a portion of the first lipid or the second lipid but not matching the tail of the PEG-linked third lipid,

wherein the liposome composition is adapted to laterally separate, via lipid phase separation, the PEG-linked third lipid from the targeting ligand-linked fourth lipid when the liposome composition is exposed to a specific environment, whereby the phase separation of the lipids exposes the targeting ligand to the more acidic environment. In one example, one of the first lipids is a zwitterionic lipid with one type of tail and one of the second lipids is a titratable head group lipid with a different type of tail. It is understood that additional lipids also can be incorporated into the composition.

In one embodiment, the liposomes can contain a targeting ligand attached to the surface of the PEG-coated liposomes. The targeting ligand can attach to the liposomes by direct attachment to liposome lipid surface components or through a short spacer arm or tether, depending on the nature of the moiety. A variety of methods are available for attaching molecules, for example, affinity moieties, to the surface of lipid vesicles. In one method, the targeting ligand is coupled to the lipid by a coupling reaction described below in the Examples, to form a targeting ligand-lipid conjugate, which conjugate is added to a solution of lipids for formation of liposomes. In another illustrative method, a vesicle-forming lipid activated for covalent attachment of a targeting ligand is incorporated into liposomes. The formed liposomes are exposed to the targeting ligand to achieve attachment of the targeting ligand to the activated lipids. One of ordinary skill in the art can select a method to attach a targeting ligand to the liposomes without undue experimentation.

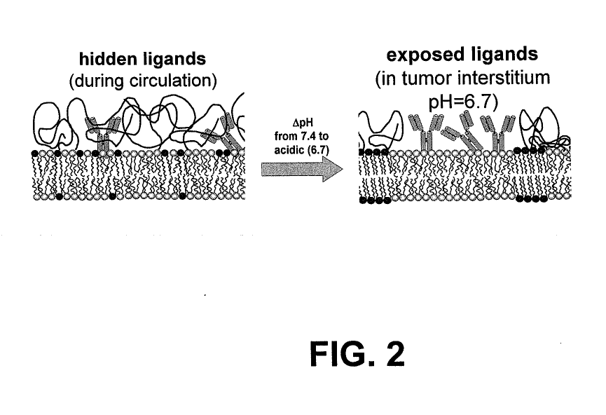

In another embodiment, the composition can selectively expose the targeting ligand to cancer cells (FIG. 2). For example, the targeting ligand is sterically obstructed by the neighboring PEG-linked lipids within the composition at a physiological (neutral) pH so that the composition can circulate in the blood steam (FIG. 2, left). As the liposome composition encounters the environment proximal to the tumor cell, which typically has a lower pH, the liposome lipid membrane forms lipid-separated domains, the neighboring PEG-linked lipids preferentially partition in lipid domains that are different from the lipid domains in which the ligand-linked lipids preferentially partition, and the targeting ligand is exposed to the tumor cell (FIG. 2, right). The exposed targeting ligand then may bind the tumor cell and deliver the biologically active agent.

Liposomes suitable for use in the composition include those composed primarily of vesicle-forming lipids. Such a vesicle-forming lipid is one that (a) can form spontaneously into bilayer vesicles in water, as exemplified by the phospholipids, or (b) is stably incorporated into lipid bilayers, with its hydrophobic moiety in contact with the interior, hydrophobic region of the bilayer membrane, and its head group moiety oriented toward the exterior, polar surface of the membrane. Many lipids suitable with this embodiment are of the type having two hydrocarbon chains, typically acyl chains, and a head group, either polar or charged. There are a variety of synthetic lipids and naturally forming lipids, including the phospholipids, such as DPPC, and DSPS (and DSPA), where the two hydrocarbon chains are typically at least 16 carbon atoms in length. The vesicle-forming lipids of this type are preferably ones having two hydrocarbon chains, typically acyl chains, and a head group, either polar or charged.

The pH-sensitive liposome can be selected to achieve a specified degree of fluidity or rigidity, to control the stability of the liposome in serum, to control the conditions effective for insertion of the targeting conjugate, to control the rate of ligand exposure for binding, and to control the rate of release of the entrapped biologically active agent in the liposome. Liposomes having a more rigid lipid bilayer, or a gel phase bilayer, are achieved by incorporation of a relatively rigid lipid, e.g., a lipid having a relatively high phase transition temperature, e.g., above about 39° C. Rigid, i.e., saturated, lipids contribute to greater membrane rigidity in the lipid bilayer. Other lipid components, such as cholesterol, are also known to contribute to membrane rigidity in lipid bilayer structures. In contrast, lipid fluidity can be achieved by incorporation of a relatively fluid lipid, typically one having a lipid phase with a relatively low liquid to gel crystalline phase transition temperature, e.g., at or below working temperature (e.g. body temperature).

These liposomes can contain titratable domain-forming lipids that phase-separate in the plane of the membrane as a response to decreasing pH values resulting in pH-controlled exposure of binding ligands for controlled targeting. In one embodiment, the liposomes are comprised of two lipid types (both T_{g}>37° C.): one type is a zwitterionic rigid lipid (e.g., dipalmitoyl phosphatidyl choline, DPPC, T_{g}=41° C.), and the other component is a ‘titratable domain-forming’ rigid lipid (e.g. distearoyl phosphatidylserin, DSPS, T_{g}=68° C.) that is triggered to phase-separate in the plane of the membrane as a response to decreasing pH values. At physiological pH (7.4) the lipid-headgroups of the ‘domain-forming’ rigid lipid (DSPS) are charged, electrostatic repulsion should prevail among DSPS lipids, and the liposomal membrane would appear more mixed and homogeneous, resulting in steric hindrance to binding of the ligand-linked lipids by the PEG-linked lipids, and in stable retention of encapsulated contents.

The lipid phase-separation can be tuned by introducing a titratable charge on the headgroups of the domain-forming lipids. The extent of ionization on the headgroups of the domain-forming lipids can be controlled by using the pH to adjust the balance between the electrostatic repulsion among the headgroups and the van der Waals attraction among the hydrocarbon chains. The longer-hydrocarbon chain lipids that could phase-separate and form domains can be selected to have titratable acidic moieties on the head group (e.g., phosphatidyl serine). At neutral pH, the headgroups of these lipids are negatively charged opposing close approximation and formation of domains. As the pH is decreased, gradual head group protonation minimizes the electrostatic repulsion and lipid domains are formed.

In one embodiment, one of the lipids of the liposomes disclosed herein can have a negatively charged head group, and can have PEG-linked chains. The PEG-linked chains can help reduce the exposure of targeting ligands to other cells when in the blood stream. In this embodiment, the liposomes comprise ionizable ‘domain-forming’ (‘raft’-forming) rigid lipids that are triggered to form domains as a response to the tumor interstitial acidic pH. Domain formation (or else lateral lipid-separation) at the tumor interstitial pH can cause the targeting ligands to be ‘exposed’ due to lateral segregation of PEG-linked lipids in lipid domains that ligand-linked lipids do not preferentially partition. At physiological pH (during circulation) the lipids are charged, the liposome membrane may be ‘mixed’ so that the targeting ligands are ‘hidden’. At the acidic tumor interstitial pH (6.7-6.5), domain-forming lipids become increasingly protonated (non-ionized) and lipid domains of clustered protonated lipids can form resulting in exposure of targeting ligands. In one embodiment, the lipids can have a pK value between about 4 and about 7.

In another embodiment, one of the lipids of the liposomes disclosed herein can have a negatively charged head group, and can have PEG-linked chains. The PEG-linked chains can help reduce the likelihood of the liposome sticking to other cells when in the blood stream. In this embodiment, the liposomes comprise ionizable ‘domain-forming’ (‘raft’-forming) rigid lipids that are triggered to form domains as a response to the endosomal/lysosomal acidic pH. Domain formation (or else lateral lipid-separation) at the endosomal/lysosomal pH can cause the encapsulated contents to be released probably due to imperfections in ‘lipid packing’ around the domain ‘rim’. At physiological pH (e.g., during circulation) the contents cannot leak, as the lipids are charged and the liposome membrane may be ‘mixed’. At the acidic late endosomal/lysosomal pH (4.5-4.0), domain-forming lipids become increasingly protonated (non-ionized) and lipid domains of clustered protonated lipids can form resulting in release of encapsulated contents. In one embodiment, the lipids can have a pK value between about 3 and about 5.

In another embodiment, the liposomes disclosed herein may further comprise stabilizing agents or have an aqueous phase with a high pH. Examples of stabilizing agents are a phosphate buffer, an insoluble metal binding polymer, resin beads, metal-binding molecules, or halogen binding molecules incorporated into the aqueous phase to further facilitate retention of hydrophilic therapeutic modalities. Additionally, liposomes may comprise molecules to facilitate endocytosis by the target cells.

Liposomes can have a more rigid lipid bilayer, which can be achieved by the incorporation of a relatively rigid lipid. For example, lipids having a higher phase transition temperature tend to be more rigid. Further saturated lipids can contribute to greater membrane rigidity in the lipid bilayer. Other lipid components, such as cholesterol, are also known to contribute to membrane rigidity in fluid lipid bilayer structures.

In another embodiment, the liposomes can comprise rigid lipids (e.g. DPPC and DSPS), PEG-linked lipids and cholesterol or a cholesterol/sterol derivative. In one embodiment, liposomes were developed containing biotin-linked lipids with dipalmitoyl tails and PEG-linked lipids with distearoyl tails that contain the titratable DSPS domain-forming lipids that can be tuned to become activated at the slightly acidic conditions that corresponds to the tumor interstitial pH. Domain formation can potentially occur when both lipid constituents (both lamellar-forming) have long saturated rigid hydrocarbon-chains, but of different lengths. It has been found that using the pH-tuned domain-forming membranes is a mechanism to create tunable rigid-liposomes that will efficiently expose the otherwise ‘hidden’ tumor-targeting ligands after liposome extravasation in tumors.

In another embodiment, the ratio of DPPC to DSPS can range from about 4:1 to about 1:2, the cholesterol content can range from about 0 to 5% mole, the DSPE-PEG (2000 MW) can be equal or less than about 0.75-1.00% mole of total lipids and more than 0.25% mole of total lipids, and the biotinylated lipid can be equal or less than 1-2% mole of total lipids. In one example, rigid liposomes having DPPC (16:0), DSPS (18:0) and 5% mole cholesterol and 0.1-1.5% mole PEG (200 MW) were incubated in PBS at 37° C. at different pH values.

### Targeting Ligand

The liposomes optionally can be prepared to contain surface groups, such as antibodies or antibody fragments, small effector molecules for interacting with cell-surface receptors, antigens, and other like compounds, for achieving desired target-binding properties to specific cell populations. Such ligands can be included in the liposomes by including in the liposomal lipids a lipid derivatized with the targeting molecule, or a lipid having a polar-head chemical group that can be derivatized with the targeting molecule in preformed liposomes. Alternatively, a targeting moiety can be inserted into preformed liposomes by incubating the preformed liposomes with a ligand-polymer-lipid conjugate. In one embodiment, the affinity molecule can be a complete antibody rather than a fragment of the antibody. While advances in antibody engineering can be employed to decrease immunogenic responses by the development of antibody fragments, tumor binding uptake and retention but for smaller fragments (Fab′, scFv) can decrease compared to the complete antibodies. These interactions can contribute to toxicities in vivo. These liposomes that are tuned to ‘hide’ antibodies during circulation and ‘expose’ the targeting ligands only in the close vicinity of cancer cells (within the acidic tumor-interstitium) can effectively address the issue of toxicity, and can reduce the issue of lower binding avidity of antibody fragments. By using the complete antibody, it is possible to achieve improved adhesion between the tumor cells and the liposomes.

Lipids can be derivatized with the targeting ligand by covalently attaching the ligand to the headgroup of a vesicle-forming lipid or to a short molecule (spacer arm or tether) already attached to the headgroup of a vesicle-forming lipid. There are a wide variety of techniques for attaching a selected ligand to a selected lipid headgroup. See, for example, Allen, T. M., et al., Biochemicia et Biophysica Acta 1237:99-108 (1995); Zalipsky, S., Bioconjugate Chem., 4(4):296-299 (1993); Zalipsky, S., et al., FEBS Lett. 353:71-74 (1994); Zalipsky, S., et al., Bioconjugate Chemistry, 705-708 (1995); Zalipsky, S., in Stealth Liposomes (D. Lasic and F. Martin, Eds.) Chapter 9, CRC Press, Boca Raton, Fla. (1995), which techniques are incorporated herein.

For example, the liposomes contain a targeting ligand, that effectively can bind specifically and with high affinity to a marker or target. In one example, the target can be the epithelial growth factor receptor family (EGFR), which is a common target for cancer therapy for solid tumors. Further, the targeting ligand can be a polypeptide or polysaccharide effector molecule capable of binding a marker on solid tumor cell. Affinity moieties, suitable with this invention, can be found in current and future literature.

Other targeting ligands are well known to those of skill in the art, and in other embodiments, the ligand is one that has binding affinity to epithelial tumor cells, and which is, more preferably, internalized by the cells. Such ligands often bind to an extracellular domain of a growth factor receptor. Exemplary receptors (epitopes) on cancer cell surfaces include the epidermal growth factor receptor (EGFR), the folate receptor, the transferrin receptor (CD71), ErbB2, and the carcinoembryonic antigen (CEA). Fransson, et al., Profiling of internalizing tumor-associated antigens on breast and pancreatic cancer cells by reversed genomics, Cancer Letters 208, 235-242 (2004).

### Biologically Active Agents

In one embodiment, the liposomal encapsulation of a biologically active agent enhances the bioavailability of the modalities in cancer cells. In this embodiment, the liposome can be used to encapsulate a biologically active agent (e.g., cancer therapeutic modalities) and efficiently release the therapeutic modality in cancer cells, thus allowing toxicity to occur in the tumor cells. For example, the use of pH sensitive liposome allows more complete release of the therapeutic modalities upon endocytosis by the cancer cell and into the late endosomal or lysosomal compartment.

The liposome can have a phospholipid-membrane rigidity to improve the retention of the bioactive agent in the liposome during blood circulation. The addition of PEG-linked lipids also reduces liposome clearance, thus increasing liposome accumulation in tumors. For example, one embodiment includes a pH-sensitive liposome with rigid membranes that combine long circulation times with the release of contents in the late endosome or lysosome. Other types of pH-sensitive liposomes can include charged titratable peptides on the surface that can cause phase separation and domain formation on charged membranes.

This invention further relates to a novel liposome structure capable of carrying bioactive agents. For example, this invention provides an improved liposome formulation and a nucleic acid, which can produce high levels of gene expression and protein production. Further, targeted α-particle emitters hold great promise as therapeutic agents for targeted cancer therapy, and can be delivered by liposomes. Other bioactive agents suitable with this invention are obvious to those with ordinary skill in the art and can be researched without undue experimentation.

Other biologically active agents suitable with such liposomes include but are not limited to natural and synthetic compounds having the following therapeutic activities: anti-arthritic, anti-arrhythmic, anti-bacterial, anticholinergic, anticoagulant, antidiuretic, antidote, antiepileptic, antifungal, anti-inflammatory, antimetabolic, antimigraine, antineoplastic, antiparasitic, antipyretic, antiseizure, antisera, antispasmodic, analgesic, anesthetic, beta-blocking, biological response modifying, bone metabolism regulating, cardiovascular, diuretic, enzymatic, fertility enhancing, growth-promoting, hemostatic, hormonal, hormonal suppressing, hypercalcemic alleviating, hypocalcemic alleviating, hypoglycemic alleviating, hyperglycemic alleviating, immunosuppressive, immunoenhancing, muscle relaxing, neurotransmitting, parasympathomimetic, sympathominetric plasma extending, plasma expanding, psychotropic, thrombolytic, and vasodilating. In one illustrative example, the entrapped agent is a cytotoxic drug, that is, a drug having a deleterious or toxic effect on cells.

### Administration of Liposome Composition

Liposomes can be used as drug delivery carriers for therapy of metastatic cancer, and other inflammatory types of diseases, and also as delivery vehicles for vaccines, gene therapy, etcetera. The present invention further provides an effective vaccine vehicle capable of effective delivery, boosting antigen-immune response and lowering unwanted extraneous immune response, presently experienced with adjuvants. The liposomes according to the invention may be formulated for administration in any convenient way. The invention therefore includes within its scope pharmaceutical compositions comprising at least one liposomal compound formulated for use in human or veterinary medicine. Other routes of administration will be known to those of ordinary skill in the art and can be readily used to administer the liposomes of the present invention.

Another embodiment of this invention includes a method comprising pre-injecting the individual with empty liposomes and saturating the reticuloendothelial organs to reduce non-tumor specific spleen and liver uptake of the liposome-encapsulated therapeutics upon administration thereof.

In use and application, the liposome can be used to preferentially deliver a biologically active agent to a target cell or cancer cell of vascularized (solid) tumors. For example, in drug delivery to metastatic tumors with developed vasculature, the preferential tumor accumulation and retention of liposomes is primarily dependent on their size (EPR effect), and can result in adequate tumor adsorbed doses that can be further enhanced by ‘switching on’ the specific targeting of cancer cells after liposome extravasation into the tumor interstitium.

The liposome of the invention may be formulated for parenteral administration by bolus injection or continuous infusion. Formulation for injection may be presented in unit dosage form in ampoules, or in multi-dose containers with an added preservative. The compositions may take such forms as suspensions, solutions or emulsions in oily or aqueous vehicles, and may contain formulatory agents such as suspending, stabilizing, and/or dispersing agents. Alternatively, the active ingredient may be in powder form for reconstitution with a suitable vehicle, e.g., sterile pyrogen-free water, before use.

One embodiment of the invention includes a method for administering a biologically active agent comprising selecting a liposome comprising at a least first rigid lipid and a second rigid lipid each having a head group and a hydrophobic tail, wherein the lipids when both protonated are not particularly miscible, and a polyethyleneglycol-linked lipid having a side chain matching at least a portion of the first or the second lipid, wherein the first lipid is a zwitterionic lipid and the second lipid has a titratable head group; and the composition is adapted to ‘expose’ targeting ligands at a certain pKa, and to release an entrapped biologically active agent at a certain pKa of lower value; preparing a liposome composition with the at least the first rigid lipid and the second rigid lipid and the polyethyleneglycol-linked lipid; preparing a therapeutic liposome by combining the composition with the biologically active agent so that the biologically active agent is within the liposome composition whereby the therapeutic liposome is adapted to release the entrapped biologically active agent at a certain pKa of lower value; and administering the therapeutic liposome to a subject.

The liposomes according to the invention may be formulated for administration in any convenient way. The invention therefore includes within its scope pharmaceutical compositions comprising at least one liposomal compound formulated for use in human or veterinary medicine. Such compositions may be presented for use with physiologically acceptable carriers or excipients, optionally with supplementary medicinal agents. Conventional carriers can also be used with the present invention.

### Overcoming Immune Response

To overcome immunogenicity, in one embodiment of the invention the liposomes are modified with PEG-linked lipids for use with the specific organism. In another embodiment, a method further comprises coating the outer membrane surfaces of the liposomes with molecules that preferentially associate with a specific target cell. These molecules or targeting agents may be antibodies, peptides, engineered molecules, or fragments thereof.

For example, to achieve tumor targeting of ovarian and breast cancer cells and internalization, liposomes can be coated (immunolabeled) with Herceptin, a commercially available antibody that targets antigens that are over-expressed on the surface of such cancer cells. Herceptin is chosen to demonstrate proof of principle with the anticipation that other antibodies, targeting ovarian, breast, liver, colon, prostate and other carcinoma cells could also be used. The target cells may be cancer cells or any other undesirable cell. Examples of such cancer cells are those found in ovarian cancer, breast cancer or metastatic cells thereof. The active targeting of liposomes to specific organs or tissues can be achieved by incorporation of lipids with monoclonal antibodies or antibody fragments that are specific for tumor associated antigens, lectins, or peptides attached thereto.

Because the biologically active agent is sequestered in the liposomes, targeted delivery is achieved by the addition of peptides and other ligands without compromising the ability of these liposomes to bind and deliver large amounts of the agent. The ligands are added to the liposomes in a simple and novel method. First, the lipids are mixed with the biologically active agent of interest. Then ligands either chemically become conjugated on the head groups of some of the lipids or ligand-linked lipids are added directly to the liposomes.

For other biologically active agents that need to be actively loaded into preformed liposomes, decoration of liposomes with targeting ligands can occur either before loading of preformed liposomes with the biologically active agents or after.

### Preparing Liposomes

The liposomes may be prepared by a variety of techniques, such as those detailed in Lasic, D. D., Liposomes from Physics to Applications, Elsevier, Amsterdam (1993), which techniques are incorporated herein. Specific examples of liposomes prepared in support of the present invention will be described herein. Typically, the liposomes can be formed by simple lipid-film hydration techniques. In this procedure, a mixture of liposome-forming lipids of the type detailed above dissolved in a suitable organic solvent is evaporated in a vessel to form a thin film, which is then covered by an aqueous medium. The lipid film hydrates have sizes between about 0.1 to 10 microns.

After formation, the liposomes are sized. One more effective sizing method for liposomes involves extruding an aqueous suspension of the liposomes through a series of polycarbonate membranes having a selected uniform pore size in the range of 0.03 to 0.2 micron, typically about 0.05, 0.08, 0.1, or 0.2 microns. The pore size of the membrane corresponds roughly to the largest sizes of liposomes produced by extrusion through that membrane, particularly where the preparation is extruded two or more times through the same membrane. Homogenization methods are also useful for down-sizing liposomes to sizes of 100 nm or less. In one embodiment of the present invention, the liposomes are extruded through polycarbonate filters with pore size of 0.1 μm resulting in liposomes having diameters in the approximate range of about 120 nm.

Incorporating Biologically Active Agent into Liposomes

The biologically active agent of choice can be incorporated into liposomes by standard methods, including passive entrapment of a water-soluble compound by hydrating a lipid film with an aqueous solution of the agent, passive entrapment of a lipophilic compound by hydrating a lipid film containing the agent, and loading an ionizable drug against an inside/outside liposome pH gradient. Other methods, such as reverse evaporation phase liposome preparation, are also suitable.

Another embodiment includes a method of formulating a therapeutic liposome composition having sensitivity to a target cell. The method includes selecting a liposome formulation composed of pre-formed liposomes comprising at least a first lipid and a second lipid each having a head group and a hydrophobic tail, wherein the lipids when both protonated are not particularly miscible, and containing PEG-linked lipids of one type of tails, and having an entrapped biologically active agent; selecting from a plurality of targeting conjugates a targeting conjugate composed of a lipid having a polar head group and a hydrophobic tail of the other type than that of the PEG-linked lipid's, and a targeting ligand attached to the headgroup of the lipid; and combining the liposome formulation and the selected targeting conjugate to form a therapeutic, target-cell pH sensitive liposome composition.

### Kits

The present invention includes kits containing the present liposome structure capable of carrying a reagent within it. One such kit may comprise the liposome structures ready for the user to add the biological reagent of interest. A kit may further comprise a liposome preparation and one or more specific biologically-active reagents for addition to the liposome structure. Another kit of the present invention comprises a set of liposome structures, each containing a specific, biologically-active reagent, which when administered together or sequentially, are particularly suited for the treatment of a particular disease or condition.

### EXAMPLES

### Example 1

Biotinylated liposomes (1% mole DPPE-biotin) were developed containing PEGylated lipids that contain domain-forming lipids, which were tuned to become activated at conditions similar to those of tumor interstitial pH. Rigid liposomes consisting of DPPC (16:0), DSPS (18:0) (at 1:1 mole ratios), and 5% cholesterol and 0.1 to 1.5% mole DSPE-PEG (2000 MW) were incubated in PSB at 37° C. at various pH values.

### Example 2

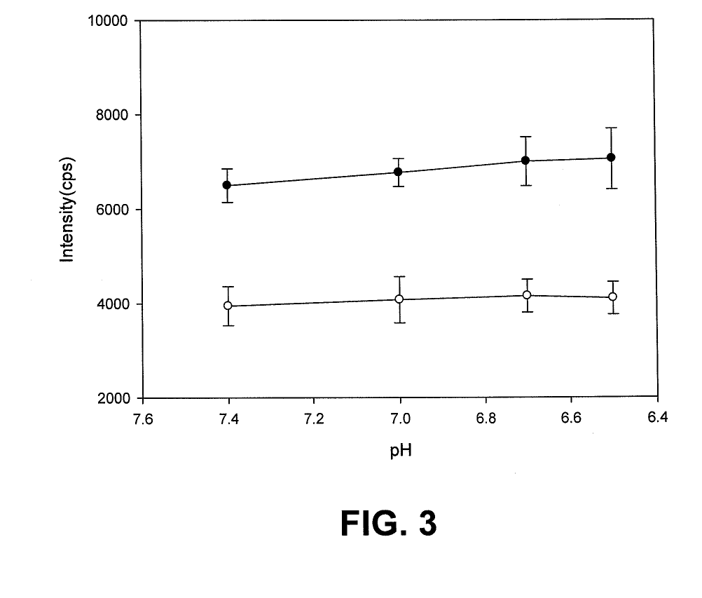

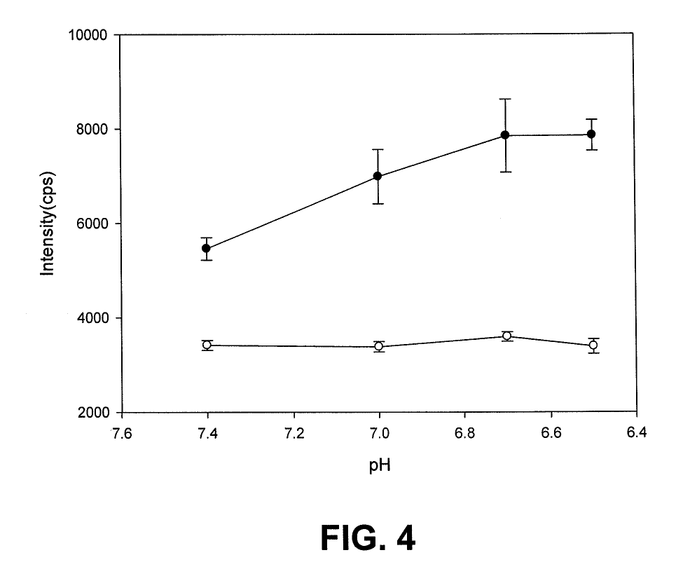

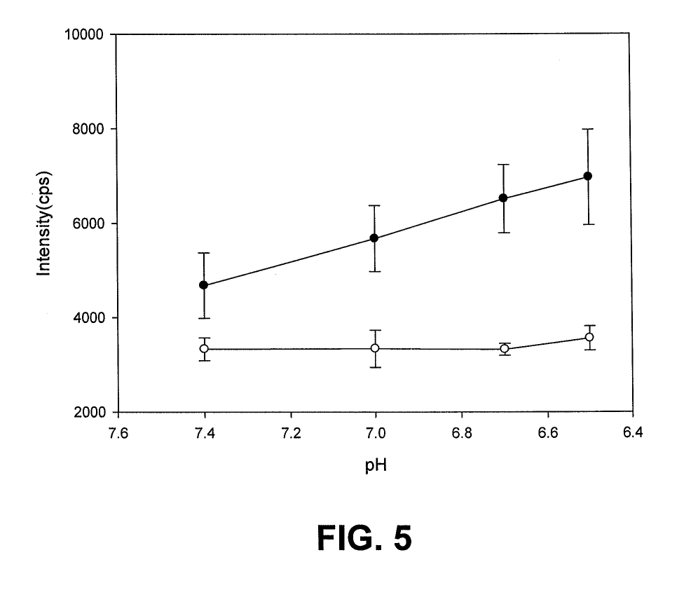

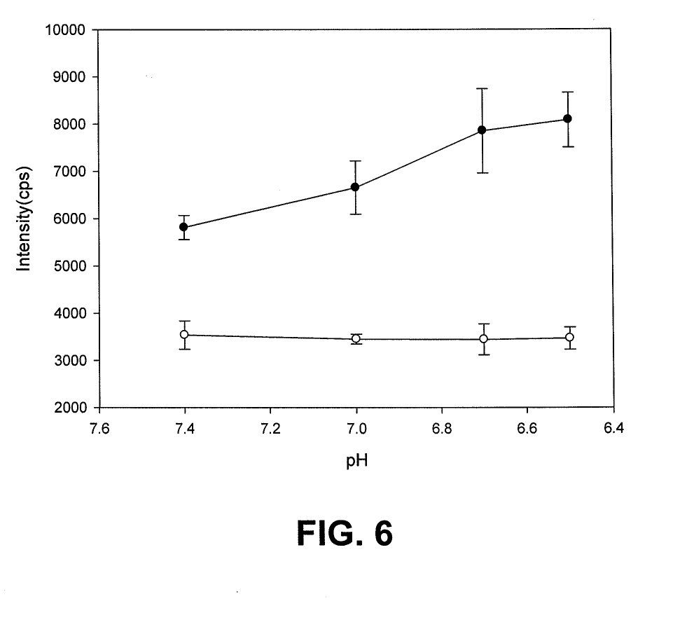

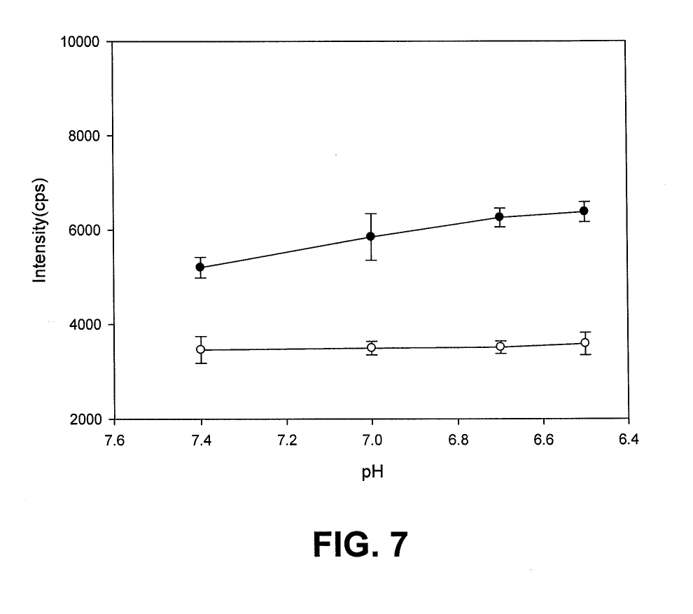

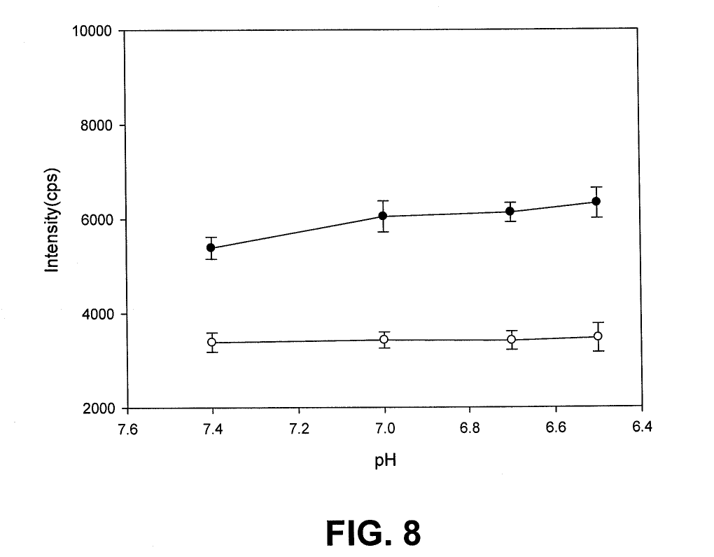

Binding of rigid biotinylated liposomes (FIGS. 3, 4, 5, 6, 7, 8, filled symbols) to streptavidin-covered—magnetic microbeads was evaluated at various pH values ranging from pH 7.4, approximating the pH of the blood during circulation of liposomes to pH 6.5 that corresponds to the pH of the tumor interstitium after extravasation of liposomes into the tumor. The extent of liposomes bound was evaluated for different amounts of PEG-linked lipids in the liposome composition ranging from 0.1 to 1.5% mole (of total lipid) and was also compared to identical liposomes without biotin (plain liposomes) indicated by the open symbols in FIGS. 3, 4, 5, 6, 7, and 8. In biotinilated liposomes the amount of biotin-linked lipids was retained constant at 1% mole of total lipid (FIG. 3 shows liposomes containing 0.1% mole PEG-linked lipid, FIG. 4 0.25% mole, FIG. 5 0.5% mole, FIG. 6 0.75% mole, FIG. 7 1.0% mole, and FIG. 8 1.5% mole). The liposomal membrane was labeled with rhodamine, and liposomes were allowed to bind to the magnetic beads and after ten successive magnetic separations and washings with PBS, the magnetic beads were incubated in fresh PBS (pH=7.4) with Triton-X 100 to release the bound lipids that were then quantitated by measuring their fluorescence intensity. An increase in fluorescence intensity (cps), with a decrease in the pH of the incubation environment during binding, showed that the affinity marker or target ligand was exposed in the lower pH environment.

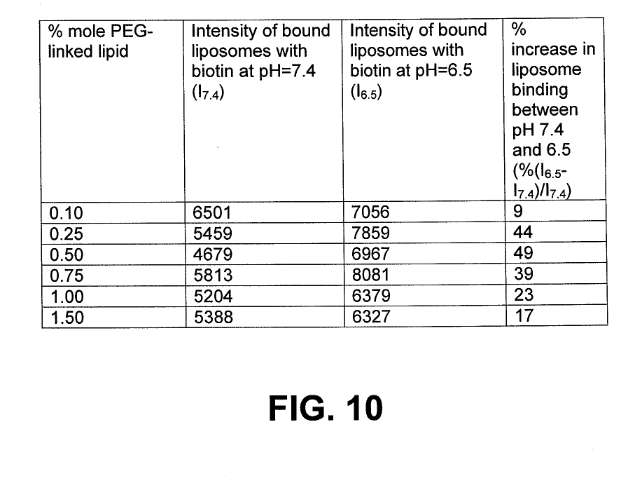

For fractions of PEG-linked lipids ranging between 0.25 and 0.75% mole, the specific binding efficacy shows a sharp transition within the narrow pH values of the physiological pH=7.4 and the tumor interstitial pH=6.7 (FIGS. 4, 5, 6). Table 1 or FIG. 10 shows the calculated fractional increase in biotinylated-liposome binding between pH 7.4 and pH 6.5 for all PEG-linked lipid compositions using the following formula: (I_{6.5}−I_{7.4})/I_{7.4}×100. The maximum increase in binding between pH 7.4 and pH 6.5 for liposomes containing biotin-linked lipids as the target ligands is observed for 0.5% mole PEG-linked lipids that was measured to be 49% (Table 1 or FIG. 10).

Depending on the molecular size (length) of the targeting ligand (defined as the distance that the binding moiety extends from the physical surface of the liposome, using trial and error, the fraction of PEG-linked lipids that have to be included in the lipid composition to maximize the increase in specific binding between pH 7.4 and 6.5. To optimize the conditions for maximum binding between different pH values, the pKa of the ionized titratable lipid was adjusted.

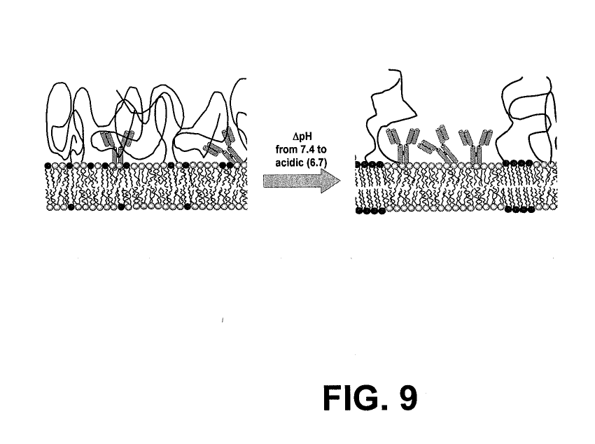

In particular, at low mole fractions of PEGylated lipids (from 0.25% to 0.75% mole for the particular example above), the PEG-chains are most probably in the “mushroom” regime and their ‘effective’ size is comparable to the size of biotin, thus upon domain formation the clustered biotin molecules are available to bind. At high mole fractions of PEGylated lipids (1% and 1.5% mole as shown on the above example), the PEG-chains become gradually extended with ‘effective lengths’ that should be ‘taller’ than the grafted biotin (as suggested in FIG. 9), thus even after domains form (for relatively small domain sizes) biotins are still ‘hindered’ by the extended PEG-chains to bind to the target (FIG. 9) de Gennes, P. G. Conformations of polymers attached to an interface, Macromolecules 13, 1069-1075 (1980). These results demonstrate that at low PEG grafting densities (0.25, 0.5 and 0.75% mole) liposome binding is pH dependent and it increases with decreasing pH. The pH determines the lateral lipid phase separation and domain formation within the liposome membranes (due to selective protonation of anionic lipids). Lipid domains, upon formation, pull the PEG-linked lipids away from the ligand-linked lipids and allow ligand specific targeting if the effective length of PEG chains above the physical surface of liposome is not significantly longer than the length of targeting ligands. For the particular lipid composition (equimolar DPPC and DSPS lipids) the most sensitive pH-dependent binding behavior is exhibited by the 0.5% mole PEGylated liposomes that shows 49% increase in binding between pH 7.4 and pH 6.5. At very low PEG grafting densities (0.1% mole) liposomes bind extensively at every pH value since not adequate steric hindrance is provided by the polymer chains that hardly cover the liposome surface (FIG. 9). At higher PEG grafting densities (1.0% and 1.5% mole, FIG. 7 and FIG. 8, respectively) the pH-dependent binding response is lost due to the extended effective length of the PEG chains that is loner than the size of the targeting ligand used in this example.

### Example 3

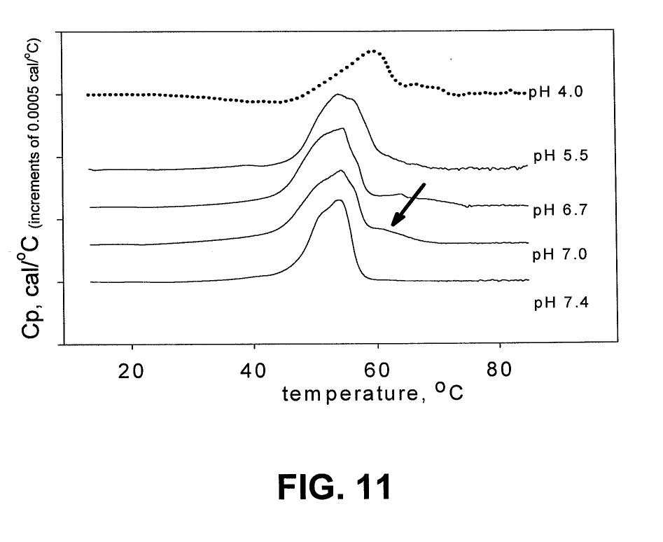

Differential Scanning Calorimetry (DSC) was used because it can provide direct evidence of phase separation of lipid membranes. FIG. 11 shows the thermal scans of the same liposome composition (equimolar DPPC and DSPS with 5% mole Cholesterol and 2% mole DSPE-PEG), performed at a rate of 60° C./h. As the pH was decreased from 7.4 to 4.0, an enhancement was observed on the contributions from thermal transitions at higher temperatures. Higher thermal transitions at lower pH values suggest increasing formation of lipid phases that are rich in clustered (protonated) DSPS lipids (that has higher Tg) and phases poor in DSPS lipids (or richer in DPPC lipids, FIG. 11). These results demonstrate that in membranes containing lipids with different hydrocarbon chain lengths (with one lipid type bearing charged headgroups), lipid mixing or domain formation is controlled by the pH that affects the extent of electrostatic repulsion among the titratable lipids.

### Example 4

The release of encapsulated fluorescent contents, specifically in this example calcein, from PEGylated liposomes, composed of equimolar ratios of DPPC and DSPS was investigated by calcein quenching efficiency measurements. The lipid film was hydrated in 1 ml phosphate buffer containing 55 mM calcein (pH 7.4, isosmolar to PBS). The unentrapped calcein was removed at room temperature by size exclusion chromatography (SEC) using a Sephadex G-50 column (of 11 cm length) and was eluted with phosphate buffer (1 mM EDTA, pH=7.4). To evaluate the release of calcein from the liposomes, the liposomes containing self-quenching concentrations of calcein (55 mM) were incubated in phosphate buffer at different pH values at 37° C. over time. The concentration of lipids for incubation was 0.20 μmoles/ml.

The release of calcein from the liposomes and its dilution in the surrounding solution resulted in an increase in fluorescence due to relief of self-quenching. Calcein release was measured at different time points by adding fixed quantities of liposome suspensions into cuvettes (1 cm path length) containing phosphate buffer (1 mM EDTA, pH 7.4). Calcein fluorescence (ex: 495 nm, em: 515 nm) before and after addition of Triton-X 100, was measured using a Fluoromax-2 spectrofluorometer (Horiba Jobin Yvon, N.J.), and was used to calculate the quenching efficiency defined as the ratio of fluorescence intensities after and before addition of Triton-X 100. The percentage of retained contents with time was calculated as follows:

$$\% calcein retention = ( \frac{{Q}_{t} - {Q}_{min}}{{Q}_{max} - {Q}_{min}} ) \times 100$$

where, Qₜ is calcein quenching efficiency at the corresponding time point t, Qₘₐₓ is the maximum calcein quenching efficiency in phosphate buffer (at pH 7.4) at room temperature immediately after separation of liposomes by SEC, and Qₘᵢₙ is the minimum quenching efficiency equal to unity.

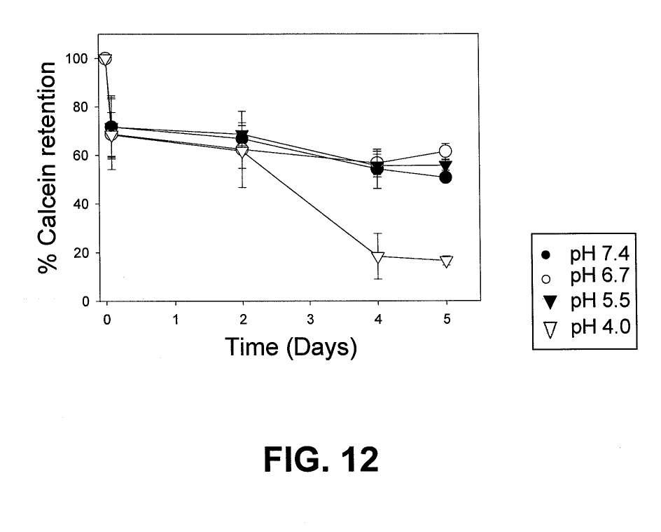

FIG. 12 shows the percentage of calcein retention as a function of pH {pH 7.4 (), pH 5.5 (◯), pH 5.0 (▾), pH 4.0 (∇)} by liposomes composed of equimolar DPPC and DSPS (with 5% mole cholesterol and 2% mole PEGylated lipids), incubated in PBS at 37° C. FIG. 5 shows the content release over 5 days. The error bars correspond to standard deviations of repeated measurements of two liposome preparations, two samples per preparation per time point. The initial drop in content retention during the first 10 minutes of incubation is probably due to osmotic and temperature differences between the encapsulated and surrounding solutions. After the first 10 minutes, encapsulated contents are stably retained by liposomes, and effectively released at the acidic pH=4 that corresponds to late endosomal lysosomal values, indicating that these liposomes can effectively release their therapeutic cargo after specific binding and endosomal internalization by target cells.

The foregoing detailed description of the preferred embodiments and the appended figures have been presented only for illustrative and descriptive purposes. They are not intended to be exhaustive and are not intended to limit the scope and spirit of the invention. The embodiments were selected and described to best explain the principles of the invention and its practical applications. One skilled in the art will recognize that many variations can be made to the invention disclosed in this specification without departing from the scope and spirit of the invention.

What is claimed is:

1. A liposome composition containing a biologically active agent, comprising:
a) at least two lipid phase separated domains formed by
i) a first lipid having a head group and a hydrophobic tail that, when protonated, is substantially miscible, wherein the first lipid is a zwitterionic lipid;
ii) a second lipid having a titratable charged head group, and a hydrophobic tail that, when protonated, is substantially immiscible with the first lipid;
b) a third lipid having a tail matching at least a portion of the first lipid or the second lipid, wherein the third lipid is PEG-linked; and
c) a targeting ligand capable of binding an antigen or a marker and linked to the headgroup of a fourth lipid having a tail matching at least a portion of the first lipid or the second lipid but not matching the tail of the PEG-linked third lipid, wherein the liposome composition is adapted to laterally separate, via lipid phase separation, the PEG-linked third lipid from the targeting ligand-linked fourth lipid when the liposome composition is exposed to a specific environment,
whereby the phase separation of the lipids exposes the targeting ligand to the more acidic environment.

2. The composition as claimed in claim 1, wherein the specific environment is an acidic environment.

3. The composition as claimed in claim 1, wherein the composition liposomes having a phase transition temperature above 37° C.

4. The composition as claimed in claim 1, wherein the biologically active agent is selected from the group consisting of chemotherapeutics, radionuclides, nucleotide fragments, and peptide fragments.

5. The composition as claimed in claim 1, wherein the targeting ligand is exposed when the composition is in an environment having a pH between about 6.3 and about 6.7.

6. The composition as claimed in claim 1, wherein the antigen or receptor is expressed at higher concentration in cancer cells than in non-cancer cells.

7. The composition as claimed in claim 1, wherein the targeting ligand is an antibody or fragment thereof.

8. The composition as claimed in claim 1, wherein the targeting ligand is an antibody fragment wherein the phase separation of the lipids physically pull the PEG-linked lipid away from ligand linked lipids in the physical surface of the liposome membrane.

9. A method for treating a malignant tumor in a mammal comprising administering to the mammal a pharmaceutically effective amount of the liposome composition of claim 1.

10. The method as claimed in claim 9, wherein the mammal is a human.

11. The composition as claimed in claim 1, wherein the liposomes bear a negative charge at a neutral pH.

12. A method for increasing accumulation of a biologically active agent proximal to a cell having a acidic environment, comprising
administering the composition of claim 1 whereby the phase separation of the lipids exposes the targeting ligand to the more acidic environment, the liposome travels to the acidic environment, exposes the targeting ligand in the acidic environment, specifically binds to the target cells, becomes endocytosed by the targeted cells, and releases the entrapped biologically active agent in the acidic environment of the late endosomal/or lysosomal compartment.

13. The method as claimed in claim 12, wherein the first lipid is DPPC and the second lipid is DSPS.

14. The method as claimed in claim 12, wherein the first lipid is DPPC and the second lipid is DSPS, and wherein 0 to 5% cholesterol is included in the composition.

15. The method as claimed in claim 12, wherein the liposomes are administered via injection.

16. The method as claimed in claim 12, wherein the liposomes are prepared to preferentially bind tumor cells at a pH of about 6.7 to about 6.5.

17. The method as claimed in claim 12, wherein the liposomes are prepared to release the biologically active agent in an environment with a pH of less than less about 6.

18. A composition containing a biologically active agent, comprising:
a) anionic liposomes having liquid phase separated domains that phase separate at a certain pH;
b) a targeting ligand capable of binding to an antigen or a marker on a tumor cell and linked to the headgroup of a lipid,
whereby the lipid-phase separation exposes the targeting ligand to the environment.

19. The composition as claimed in claim 18, wherein the liposome has at a least first lipid and a second lipid each having a head group and a hydrophobic tail; at least at least two types of lipid tails, which when both protonated, are not substantially miscible and form more than one lipid phase-separated domains.

20. The composition as claimed in claim 18, wherein the liposome further comprises a polyethyleneglycol-linked lipid having a tail matching at least a portion of the first or the second lipid.

21. The composition as claimed in claim 18, wherein the composition is adapted to laterally separate the PEG-linked lipids from the targeting ligand-linked lipids on the plane of the membrane after liposomes are exposed to an environment with a decrease pH.

22. A liposome composition containing a biologically active agent, comprising:
a) at least two lipid phase separated domains formed by
i) a first lipid having a head group and a hydrophobic tail that, when protonated, is substantially miscible, wherein the first lipid is a zwitterionic lipid;
ii) a second lipid having a titratable charged head group, and a hydrophobic tail that, when protonated, is substantially immiscible with the first lipid;
b) a targeting ligand capable of binding an antigen or a marker and linked to the head group of a third lipid having a tail matching at least a portion of the first lipid or the second lipid, wherein the liposome composition is adapted to laterally separate, via lipid phase separation, when the liposome composition is exposed to a specific environment,
whereby the phase separation of the lipids exposes the targeting ligand to a more acidic environment.

23. The composition as claimed in claim 1, wherein the first lipid and the second lipid when the second lipid is electrostatically charged are less immiscible with each other and form less lipid phase-separated domains.
